(19) United States
(12) Patent Application Publication
(10) Pub. No.: US 2010/0100047 A1
(43) Pub. Date: Apr. 22, 2010

(54) MEDICATION DELIVERY DEVICE WITH PRE-SET BOLUS PUMP

(75) Inventors: Kristian Glejbøl, Glostrup (DK); Frank Daniel, Hellerup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/529,450

(22) PCT Filed: Feb. 29, 2008
(86) PCT No.: PCT/EP08/52484

§ 371 Date: Jan. 4, 2010

(30) Foreign Application Priority Data
Mar. 2, 2007 (EP) 07103394.8

(51) Int. Cl. A61M 25/06

(52) U.S. Cl. 604/171

(57) ABSTRACT

The present invention relates to a handheld medication delivery device comprising first and second mutually displaceable housing parts (1, 2) optionally being operatively connected by a first resilient member thereby allowing the medication delivery device to be in a non-compressed state and in a compressed state. The first housing part (2) is adapted to house a collapsible reservoir containing a medicament, whereas one of the housing parts is arranged to form a protective shield around an associated hypodermic needle (5) when said medication delivery device is in a non-compressed state

### FIELD OF THE INVENTION

The present invention relates to a medication delivery device comprising a pre-set bolus pump. Also, the present invention relates to a medication delivery device where the amount of medicament to be expelled from the medication delivery device may be selected among a relatively few numbers of pre-selected amounts of medicament. In this way a simple and very reliable medication delivery device can be provided.

### BACKGROUND OF THE INVENTION

Various types of medication delivery devices have been suggested over the years. Many of these devices are advanced and sophisticated medication delivery devices being capable of delivering adjustable amounts of medicament, such as insulin, over a large dosage range and with high dosage accuracy.

However, some modern drugs do not require a precise dosage setting as the therapeutic window for these drugs is relatively large. An example of this is treatment of diabetes mellitus using GLP-1 or GLP-1 analogues. In this situation there is no need for highly sophisticated medication delivery devices being capable of delivering medicament over a large dosage range and with high dosage accuracy.

Thus, there is a need for simplified medication delivery devices which can deliver a fixed dosage of medicament. Also, there is a need for medication delivery devices which can deliver one of a few pre-selectable dosages of medicament. Such medication delivery devices can be made simpler in their mechanical construction will be simpler and more convenient to handle during use.

It may be seen as an object of the present invention to provide a simple and reliable mechanical medication delivery device capable of expelling pre-selected amounts of medicament, such as insulin.

### SUMMARY OF THE INVENTION

The above-mentioned object is complied with by providing, in a first aspect, a handheld medication delivery device comprising
first and second mutually displaceable housing parts allowing the medication delivery device to be in a non-compressed state and in a compressed state
   wherein the first housing part is adapted to house a collapsible reservoir containing a medicament, and wherein one of the housing parts is arranged to form a protective shield around an associated hypodermic needle when said medication delivery device is in a non-compressed state.

The medication delivery device may further comprise a first resilient member operatively connected between the first and second housing parts, and wherein the second housing part is arranged to form a protective shield around the associated hypodermic needle when said medication delivery device is in a non-compressed state.

In the present context the term “collapsible” should be interpreted broadly. Thus, collapsible is to cover a reservoir comprising a flexible sheet-like material which changes its form with changes of the volume of the reservoir. In addition, the term collapsible is also to cover any arrangement which allows changes in a volume of a reservoir. Such changes in volume could be provided by moveable wall portions of the reservoir as long as the pressure inside the reservoir maintains at approximately the same level as the pressure outside the reservoir.

A pressure difference of around 0.1 bar between the interior of the collapsible reservoir and the surroundings may be acceptable. However, it is an advantage of the present invention that the interior pressure in the collapsible reservoir is kept at essentially the same level—independent of the amount of medicament in the first collapsible reservoir.

The medication contained in the collapsible reservoir may in principle be any kind of medication, such as one or more peptides, one or more proteins or a combination hereof. Thus, the peptides or proteins may comprise insulin, insulin analogues, GLP or GLP analogues or a mixture comprising one or more of these.

In the context of the present invention, “hypodermic needle” should be interpreted broadly, i.e. comprising injection needles, infusion sets, micro-needle arrays or other suitable means for mechanically penetrating the dermis, hereby allowing for infusion of a substance.

Thus, according to the first aspect of the present invention a handheld medication delivery device comprising two mutually displaceable housing parts is provided. By handheld is meant that the medication delivery device may be operated using only one hand. The first and second housing parts may be arranged along a common centre axis of the medication delivery device. Similarly, the displacements of the first and second housing parts may be along said common centre axis. In a compressed state the first or the second housing part may be at least partly displaced into a hollow section of the other housing part. Thus, in a compressed state of the medication delivery device the overall length of the medication delivery device is shortened compared to the length of the medication delivery device in its non-compressed state. The first resilient member may be a linear spring.

The first housing part of the medication delivery device may act as a handle in the form of a substantially hollow shell wherein the collapsible reservoir is positioned, whereas the second housing part may be that part of the medication delivery device which is placed on the skin of the patient to receive an amount of medicament. In this arrangement the medication delivery device is activated by positioning the second housing part on the skin and displacing the first housing part towards the skin.

The first and second mutually displaceable housing parts may be arranged so that an amount of medicament is expelled from the medication delivery device when the first and second mutually displaceable housing parts are moved towards each other. Thus, upon moving or displacing the first and second housing parts towards each other medicament may be expelled from the medication delivery device.

The medication delivery device may further comprise a loading chamber being in fluid communication with the collapsible reservoir. The medication delivery device may further comprise a piston member fixedly arranged relative to first housing part. The piston member may be a separate component attached to the first housing part, or alternatively, the piston member may form an integral part of the first housing part. The piston member may comprise a through-going conduit thereby providing a fluidic communication between the collapsible reservoir and the loading chamber. The through-going conduit of the piston member may comprise a one-way valve, said one-way valve allowing medicament to flow in a direction from the collapsible reservoir to the loading chamber only. In this way medicament is prevented from flowing from the loading chamber and back into the collapsible reservoir. The one-way valve may be implemented is various ways, for example as a septum positioned in the conduit near the loading chamber.

The medication delivery device may further comprise a displaceable member being operatively connected to the first housing part via a second resilient member, said displaceable member further being operatively connectable to the associated injection needle. The second resilient member may be a linear spring. In addition, the medication delivery device may comprise a loading chamber housing adapted to perform controlled movements relative to the displaceable member, said controlled movements being between two end stops provided by the displaceable member. Thus, the loading chamber housing may be displaceable relative to the displaceable member between two well-defined end positions. In a non-compressed state the loading chamber housing abuts one of the end stops, whereas in a compressed state of the medication delivery device the loading chamber housing abuts the other end stop. Thus, the loading chamber housing may be arranged to perform controlled movements relative to the displaceable member, said controlled movements being in response to relative movements between the displaceable member and the first housing part. The loading chamber housing may comprise an opening adapted to receive the piston member. Opposite the opening of the loading chamber a penetrable septum may be arranged. The penetrable septum allows a backend of an associated injection needle to be in fluidic communication with the loading chamber so that pressurised medicament in the loading chamber can escape via the injection needle. Pressurised medicament may be provided by displacing the piston in the direction of the penetrable septum.

The medication delivery device may further comprise a hypodermic needle, such as an injection needle, being in fluidic communication with the loading chamber when the medication delivery device is in its compressed state. The injection needle may be in a mutually fixed relationship with the displaceable member, for example by attaching the injection needle to a hub with inner threads, said inner threads of the hub engaging outer threads of the displaceable member.

The medication delivery device may further comprise a collapsible reservoir. In its simplest form the collapsible reservoir is made from sheet material which is folded and welded, thus forming a closed bag. Alternatively, the collapsible reservoir may comprise a substantially rigid portion and a collapsible portion, the substantially rigid and the collapsible portions being adapted to contact the medicament, the collapsible portion being adapted to collapse into at least part of the substantially rigid portion upon expelling medicament from the reservoir. The collapsible portion of the reservoir may comprise a sheet material, said sheet material comprising a thermoplastic material. The thermoplastic material may form part of a multilayer sheet structure. The sheet material may further comprise one or more barrier layers. Finally, the sheet material may have a thickness smaller than 1 mm, such as smaller than 0.8 mm, such as smaller than 0.5 mm, such as smaller than 0.3 mm.

In a second aspect, the present invention relates to a handheld medication delivery device for delivering a pre-selected and essentially nonadjustable amount of medicament, the medication delivery device comprising
a collapsible reservoir adapted to contain the medicament,
a loading chamber being in fluid communication with the collapsible reservoir, and
means for expelling the pre-selected amount of medicament from the loading chamber.

In a third aspect, the present invention relates to a handheld medication delivery device for delivering a pre-selected amount of medicament, the medication delivery device comprising
a collapsible reservoir adapted to contain medicament,
a loading chamber being in fluid communication with the collapsible reservoir, and
means for expelling the pre-selected amount of medicament from the loading chamber.

According to the third aspect, the pre-selected amount of medicament may be selectable among a number of pre-selectable amounts, said number of pre-selectable amounts being smaller than about 10.

In terms of implementation, the medication delivery devices according to the second and third aspects may be implemented following the same design principles as the medication delivery device according to the first aspect of the present invention.

### BRIEF DESCRIPTION OF THE DRAWINGS

The present invention will now be explained in further details with reference to the accompanying figures, wherein

FIG. 1 shows a side view of a medication delivery device according to the present invention,

FIG. 2 shows an expanded view of the needle region of a medication delivery device according to the present invention, and

FIG. 3 shows a side view of a medication delivery device in a compressed state.

While the invention is susceptible to various modifications and alternative forms, specific embodiments have been shown by way of example in the drawings and will be described in details herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

In its most general aspect the present invention relates to a medication delivery device for delivering a pre-selected and essentially nonadjustable amount of medicament, the medication delivery device comprising a collapsible reservoir adapted to contain the medicament, a loading chamber being in fluid communication with the collapsible reservoir, and means for expelling the pre-selected amount of medicament from the loading chamber.

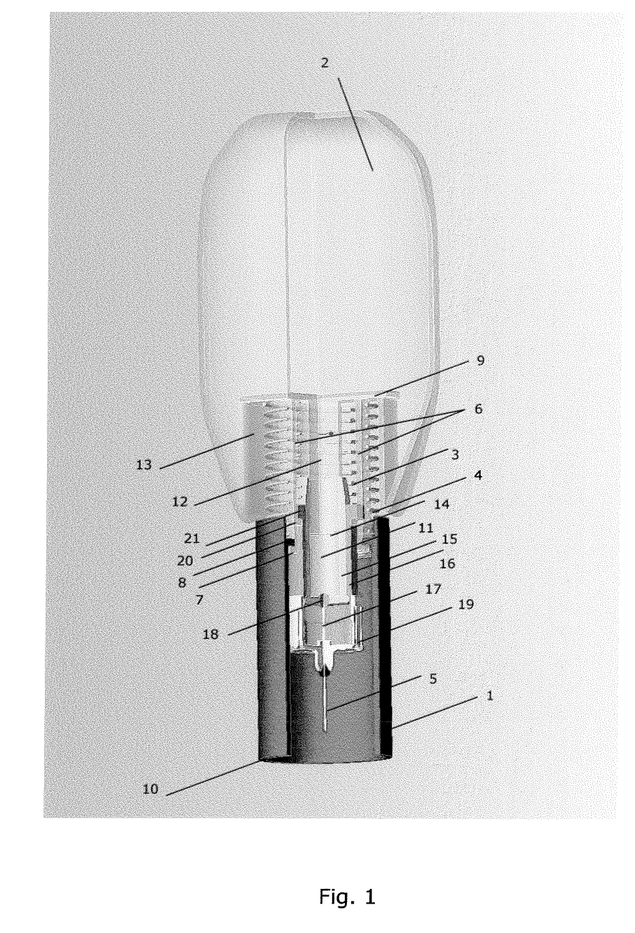

Referring now to FIG. 1 a medication delivery device according to the present invention is depicted. As seen, the medication delivery device comprises two mutually displaceable housing parts 1, 2. In FIG. 1 the medication delivery device is depicted in a non-compressed state. By compressing inner and outer linear springs 3, 4 housing parts 1, 2 may be moved towards each other. During compression medicament may be expelled from the medication delivery device.

In FIG. 1 lower housing part 1 serves as a protecting shield for injection needle 5 when the medication delivery device is in a non-compressed state. The lower housing part 1 is adapted to slide along element 6 of the upper housing part 2. In the non-compressed state the upper and lower housing parts are kept apart by outer linear spring 4. Mechanical stops 7, 8 are provided on both element 6 of the upper housing part 2 and the lower housing portion 1. Thus, mechanical stops 7, 8 define the outermost position of the lower housing part 1 relative to the upper housing part 2 when the medication delivery device is in a non-compressed state. As seen in FIG. 1 the linear outer spring 4 rests against mechanical stop 8 and surface 9 of the upper housing part 2.

The upper housing portion 2 comprises a collapsible reservoir (not shown) containing the medicament to be expelled from the medication delivery device. In its simplest form the collapsible reservoir is made from sheet material which is folded and welded, thus forming a closed bag. If this type of collapsible reservoir is employed it is normally necessary to attach some sort of coupling unit to the reservoir. Although the sheet material for a simple reservoir can be chosen from a wide range of materials, the preferred materials are thermoplastics or laminates containing at least one layer of thermoplastic material.

Another type of collapsible reservoir comprises a substantially rigid portion and a collapsible portion wherein at least a part of the rigid portion and at least a part of the collapsible portion are adapted to contact the medicament to be contained in the reservoir. The collapsible portion is adapted to collapse into at least part of the substantially rigid portion upon expelling medicament from the reservoir.

The term “collapsible” should be interpreted broadly. Thus, collapsible is to cover a reservoir comprising a flexible sheet-like material which changes its form with changes of the volume of the reservoir. In addition, the term collapsible is also to cover any arrangement which allows changes of a volume of a reservoir. Such changes in volume could be provided by moveable wall portions of the reservoir as long as the pressure inside the reservoir maintains at approximately the same level as the pressure outside the reservoir.

The sheet material of the collapsible reservoir should fulfil a number of different demands if employed for production of a reservoir. Most important is that the reservoir should have excellent barrier properties and be compatible with the medication to be stored in the reservoir. Additionally, the material should be processable, i.e. if welding is chosen as the preferred process of joining the sheet material the material should be weldable. Additionally, the material should be able to withstand the mechanical loads to which it will be subjected during processing, transport and use. A final demand often put on the sheet material is that it should be possible to sterilize the material without critical degradation.

Due to the many conflicting demands on the sheet material, the sheet material may be a multilayer structure made from two or more layers having different properties. The sheet material will often be made predominantly from a laminate of multiple thermoplastic layers having the required mechanical properties. One or more barrier layers will be sandwiched between thermoplastic layers. Among inorganic barrier layers inorganic materials like Al AlOₓ, AlₓO_{y}N_{z}, SiOₓ, SiOₓN_{y}, SiNₓ are preferred. The numbers x,y,z does not refer to any specific stochiometric composition but rather indicate a range of numbers as barrier layers often are non-stochiometric substances. Among organic barrier layers polyvinylchloride (PVC), polyparylene, cyclo olefin copolymer (COC) polypropylene (PP) and polychlorotrifluoroethylene (PCTFE) are preferred materials. Among these PP, PVC, COC and PCTFE have a high mechanical strength. They may hence be used either in a laminate or as single layer sheets.

The sheet thickness strongly depends on the stiffness and barrier properties of the sheet material. In a preferred embodiment of the present invention the average thickness of the sheet material is less than 1 mm. In a more preferred embodiment of the invention the average thickness of the sheet material is less than 0.3 mm.

Depending on the properties of the sheet material a number of different strategies for joining may be employed, including adhesive bonding, welding and mechanical joining. Among these welding, preferably laser welding, RF welding r heat welding are preferred.

Still referring to FIG. 1, with a collapsible reservoir positioned within the upper housing part 2 medicament is allows to flow into loading chamber 11 via conduit 12. Thus, in a non-compressed state of the medication delivery device loading chamber 11 is filled with medicament. The front edge 10 of the lower housing part 1 is adapted to be pushed against the skin of the patient to receive a dose of medicament. Upon positioning edge 10 of the lower housing part 1 on the skin of the patient and pushing upper housing part 2 towards the skin, lower housing part 1 is displaced into opening 13 of the upper housing part 2. At the same time piston 14, which is fixedly arranged to the upper housing part 2, provides pressure to the medicament in the loading chamber 11. Along with this, inner cylinder 15 is displaced in a forward direction relative to outer cylinder 16 causing the backend of the injection needle 17 to penetrate septum 18. When the backend of the injection needle 17 has penetrated septum 18 pressurised medicament in the loading chamber 11 is allowed to leave the loading chamber 11 via the injection needle 5, 17. As depicted in FIG. 1 the injection needle is fixedly arranged to outer cylinder 16 via hub 19.

The outer cylinder 16 is adapted to slide along element 6 of the upper housing part 2. In the non-compressed state the outer cylinder 16 and upper housing part 2 are kept apart by inner linear spring 3. Mechanical stops 20, 21 are provided on both element 6 of the outer cylinder 16. Thus, mechanical stops 20, 21 define the outermost position of the outer cylinder 16 relative to the upper housing part 2 when the medication delivery device is in a non-compressed state. As seen in FIG. 1 the linear inner spring 3 rests against mechanical stop 21 and surface 9 of the upper housing part 2.

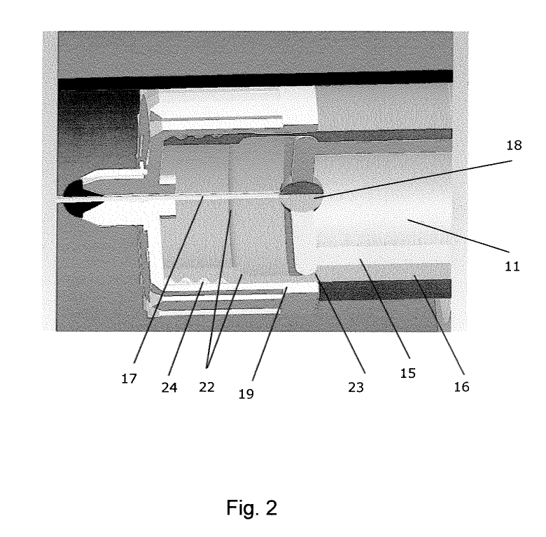

Referring now to FIG. 2 an exploded view of the injection needle portion is depicted. As seen in FIG. 2 the inner cylinder 15 is allowed to move between end stops 22 and 23 of the outer cylinder 16. Thus, in a non-compressed state inner cylinder 15 abuts end stop 23 and septum 18 forms a seal to loading chamber 11. Contrary, in a compressed state inner cylinder 15 abuts end stop 22 and septum 18 has been penetrated by the backend 17 of the injection needle. The hub 19, to which the injection needle is attached, is attached to the outer cylinder 16 via threads 24.

The medication delivery device according to the present invention is capable of delivering dosages between 0.1 and 1 ml. To deliver this the stroke length will be around 12 mm.

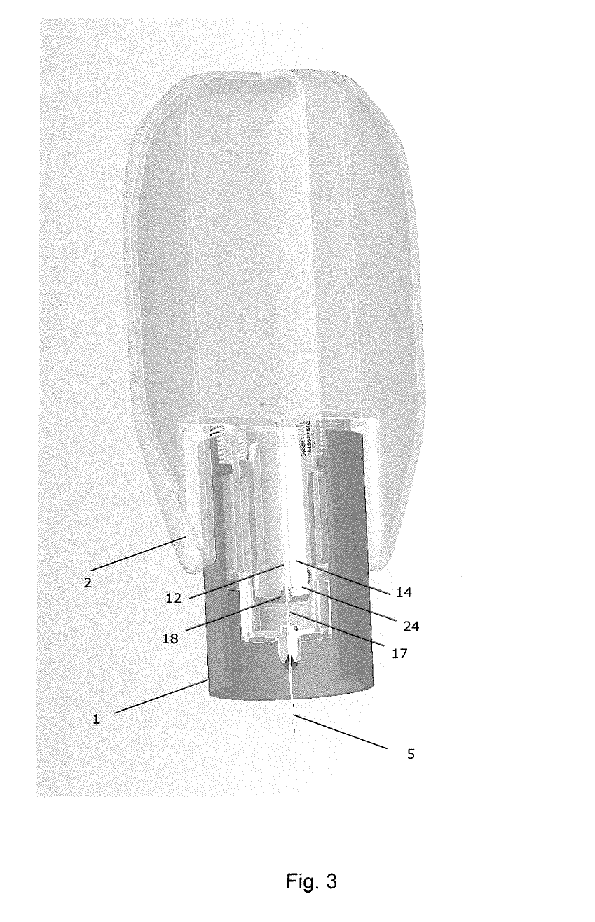

FIG. 3 depicts a medication delivery device in a compressed state. As seen in FIG. 3 the lower housing part 1 has been pressed into the upper housing part 2. A consequence of this compression is that the front end of the injection needle 5 is positioned outside the lower housing part 1. Similarly, the backend of the injection needle 17 has penetrated the septum 18 whereby medicament can leave the medication delivery device via the injection needle 5, 17. As depicted with reference numeral 24 the piston 14 abuts the bottom surface of the loading chamber when the medication delivery device is in a compressed state whereby the loading chamber is emptied via the injection needle. In order to prevent that medicament leaves the loading chamber via the conduit 12 a one-way valve (not shown) is positioned at the lower end of the conduit 12.

1. A handheld medication delivery device comprising
first and second mutually displaceable housing parts allowing the medication delivery device to be in a non-compressed state and in a compressed state
wherein the first housing part is adapted to house a collapsible reservoir containing a medicament, and wherein one of the housing parts is arranged to form a protective shield around an associated injection needle when said medication delivery device is in a non-compressed state.

2. A medication delivery device according to claim 1, further comprising a first resilient member operatively connected between the first and second housing parts, and wherein the second housing part is arranged to form a protective shield around the associated injection needle when said medication delivery device is in a non-compressed state.

3. A medication delivery device according to claim 2, wherein the first and second mutually displaceable housing parts are arranged so that an amount of medicament is expelled from the medication delivery device when the first and second mutually displaceable housing parts are moved towards each other.

4. A medication delivery device according to claim 3, further comprising a loading chamber being in fluid communication with the collapsible reservoir.

5. A medication delivery device according to claim 4, further comprising a piston member fixedly arranged relative to first housing part.

6. A medication delivery device according to claim 5, wherein the piston member comprises a through-going conduit thereby allowing fluidic communication between the collapsible reservoir and the loading chamber.

7. A medication delivery device according to claim 6, wherein the through-going conduit comprises a one-way valve, said one-way valve allowing medicament to flow in a direction from the collapsible reservoir to the loading chamber only.

8. A medication delivery device according to claim 5, further comprising a displaceable member being operatively connected to the first housing part via a second resilient member, said displaceable member further being operatively connectable to the associated injection needle.

9. A medication delivery device according to claim 8, further comprising a loading chamber housing adapted to perform controlled movements relative to the displaceable member, said controlled movements being between two end stops provided by the displaceable member.

10. A medication delivery device according to claim 9, wherein the loading chamber housing comprises an opening adapted to receive the piston member.

11. A medication delivery device according to claim 8, further comprising an injection needle being in fluidic communication with the loading chamber when the medication delivery device is in its compressed state.

12. A medication delivery device according to claim 11, wherein the injection needle is in a mutually fixed relationship with the displaceable member.

13. A medication delivery device according to claim 2, further comprising a collapsible reservoir comprising a substantially rigid portion and a collapsible portion, the substantially rigid and the collapsible portions being adapted to contact the medicament, the collapsible portion being adapted to collapse into at least part of the substantially rigid portion upon expelling medicament from the reservoir.

14. A medication delivery device according to claim 13, wherein the collapsible portion of the reservoir comprises a sheet material.

15. A medication delivery device according to claim 14, wherein the sheet material comprises a thermoplastic material.

16. A medication delivery device according to claim 15, wherein the thermoplastic material forms part of a multilayer sheet structure.

17. A medication delivery device according to claim 15, wherein the sheet material further comprises one or more barrier layers.

18. A medication delivery device according to claim 14, wherein the sheet material has a thickness smaller than 1 mm, such as smaller than 0.8 mm, such as smaller than 0.5 mm, such as smaller than 0.3 mm.
